(19) United States
(12) Patent Application Publication
(10) Pub. No.: US 2026/0251521 A1
(43) Pub. Date: Aug. 27, 2026

(54) MANAGEMENT DEVICE, MANAGEMENT SYSTEM, AUTHENTICATION DEVICE, AND MANAGEMENT METHOD

(71) Applicant: Asahi Kasei Microdevices Corporation, Tokyo (JP)

(72) Inventors: TOSHIKAZU YOSHIMINE, Tokyo (JP); TAKAAKI FURUYA, Tokyo (JP); Keiichiro Kuwata, Tokyo (JP)

(73) Assignee: Asahi Kasei Microdevices Corporation, Tokyo (JP)

(21) Appl. No.: 19/548,964

(22) Filed: Feb. 25, 2026

(30) Foreign Application Priority Data
Feb. 27, 2025 (JP) 2025-029757
Dec. 26, 2025 (JP) 2025-284624

(51) Int. Cl. G01M 3/04

(52) U.S. Cl. CPC G01M 3/04

(57) ABSTRACT

A management device includes: an environmental information acquisition part acquiring environmental information from at least one first measurement part that measures the environmental information of a measurement target; an estimation part estimating based on the environmental information a fluid leak position indicating a position where fluid leaked; and an operator identification part identifying, based on operator information indicating identification information for identifying an operator and position information related to the operator as well as the fluid leak position, an operator related to the fluid leak.

### CROSS-REFERENCE TO RELATED APPLICATION

This application claims the priority benefits of Japanese application no. 2025-029757, filed on Feb. 27, 2025, and Japanese application no. 2025-284624, filed on Dec. 26, 2025. The entirety of the above-mentioned patent application is hereby incorporated by reference herein and made a part of this specification.

### BACKGROUND

### Technical Field

The disclosure relates to a management device, a management system, an authentication device, and a management method.

### Related Art

Patent Document 1 describes an emission volume authentication application support device that monitors at least one of a gas emission volume emitted by a gas emission operator and a gas emission volume per business unit of a gas emission business site under the jurisdiction of the gas emission operator, and processes an application for emission volumes requested by the gas emission operator to an authentication organization that certifies gas emission volumes based on the monitored gas emission volume.

### CITATION LIST

### Patent Document

[Patent Document 1] Japanese Patent Application Laid-Open Publication No. 2004-152091

When a fluid leak occurs, it is desired to identify which operator caused the fluid leak and to have the result of the identification publicly authenticated.

### SUMMARY

A management device according to one aspect of the disclosure may include an environmental information acquisition part acquiring environmental information from at least one first measurement part that measures the environmental information of a measurement target. The management device may include an estimation part estimating based on the environmental information a fluid leak position indicating a position where fluid leaked. The management device may include an operator identification part identifying, based on operator information indicating identification information for identifying an operator and position information related to the operator as well as the fluid leak position, an operator related to the fluid leak.

A management system according to one aspect of the disclosure may include the management device and the at least one first measurement part.

An authentication device according to one aspect of the disclosure may include a communication interface configured to be capable of communicating with at least one first measurement part; an arithmetic device including at least one processor configured to be capable of communicating with the communication interface; and a memory device storing a predetermined authentication protocol, and the communication interface may be configured to receive environmental information, which includes concentration of at least one type of measurement target gas from the first measurement part. The arithmetic device may include an environmental information acquisition part acquiring the environmental information via the communication interface; an estimation part estimating based on the environmental information an occurrence of leak of the measurement target gas and a position where the leak occurred as a gas leak position; an operator identification part identifying, based on operator information, which includes a combination of an installation position of gas equipment and identification information of an operator managing the gas equipment as well as the gas leak position estimated by the estimation part, an operator related to the gas leak; and an authentication part authenticating based on the authentication protocol the identified operator related to the gas leak as an operator that caused the gas leak, and issuing an electronic certificate for the operator authenticated by the authentication part and transmitting the electronic certificate via the communication interface.

A management method according to one aspect of the disclosure may include: acquiring, by an environmental information acquisition part, environmental information from at least one first measurement part that measures the environmental information of a measurement target. The management method may include: estimating, by an estimation part based on the environmental information a fluid leak position indicating a position where fluid leaked. The management method may include: identifying, by an operator identification part, based on operator information indicating identification information for identifying an operator and position information related to the operator as well as the fluid leak position, an operator related to the fluid leak.

### BRIEF DESCRIPTION OF THE DRAWINGS

FIG. 1 is a diagram illustrating an example of an overall configuration of an authentication system according to the present embodiment.

FIG. 2 is a diagram illustrating an example of a simulation in which gas concentration distribution of gas diffusion in the downwind direction when a gas leak occurs is numerically calculated.

FIG. 3 is a diagram illustrating an example of functional blocks of a measurement device.

FIG. 4 is a diagram illustrating an example of functional blocks of an authentication device.

FIG. 5 is a diagram illustrating a flowchart illustrating an example of a procedure for authenticating an operator that caused a gas leak.

FIG. 6 is an example of a hardware configuration.

### DESCRIPTION OF THE EMBODIMENTS

A management device according to one aspect of the disclosure may include an environmental information acquisition part acquiring environmental information from at least one first measurement part that measures the environmental information of a measurement target. The management device may include an estimation part estimating based on the environmental information a fluid leak position indicating a position where fluid leaked. The management device may include an operator identification part identifying, based on operator information indicating identification information for identifying an operator and position information related to the operator as well as the fluid leak position, an operator related to the fluid leak.

In the management device, the environmental information may include gas concentration, and the fluid may be gas.

In any of the management devices, there may be included an authentication part authenticating the identified operator related to gas leak, which is the fluid leak, as an operator that caused the gas leak, and the management device may function as an authentication device.

In any of the management devices, in a case where the operator identification part identifies based on the operator information and the gas leak position a plurality of operators related to the gas leak, the environmental information acquisition part may further acquire environmental information including gas concentration around the gas leak position from at least one mobile second measurement part as additional environmental information. The operator identification part may identify, based further on the additional environmental information, the operator related to the gas leak from among the plurality of operators.

In any of the management devices, the operator identification part may identify an operator whose distance between a position identified by the position information and the gas leak position satisfies a predetermined distance range as the operator related to the gas leak.

In any of the management devices, in a case where a plurality of operators whose distance between the position identified by the position information and the gas leak position satisfies a predetermined distance range are identified by the operator identification part, the authentication part may authenticate that each of the plurality of operators caused the gas leak at respective ratios identified according to a magnitude of the distance between the position of the plurality of operators and the gas leak position.

In any of the management devices, there may be included a facility information acquisition part acquiring equipment information corresponding to the gas leak position by referring to equipment information indicating information related to equipment that processes the gas as the measurement target and a position of the equipment. The management device may include a facility information identification part identifying equipment information of at least one other equipment related to the equipment information corresponding to the gas leak position by referring to the equipment information.

In any of the management devices, the environmental information acquisition part may acquire environmental information around a position of the at least one other equipment from the at least one first measurement part. The estimation part may estimate, based on past environmental information around the gas leak position and the environmental information around the position of the at least one other equipment, other equipment that may cause a gas leak within a predetermined period from among the at least one other equipment.

In any of the management devices, the estimation part may estimate the gas leak position using an inverse analysis model, a statistical model, an atmospheric dispersion prediction model, or a machine learning model indicating a relationship between environmental information and a gas leak position.

In any of the management devices, the estimation part may estimate the gas leak position using an inverse analysis model, a statistical model, an atmospheric dispersion prediction model, or a machine learning model indicating a relationship between the environmental information, which includes at least one of the gas concentration, wind direction, and wind speed of gas measured by the at least one first measurement part, and an estimated gas leak position tuned based on the environmental information.

In any of the management devices, the estimation part may estimate, based on the environmental information, which includes at least one of the gas concentration, wind direction, and wind speed of gas measured by the at least one first measurement part, the gas leak position by calculating behavior of particles tracing back in time using a particle method.

In any of the management devices, the estimation part may define an objective function by comparing gas concentration at a reference point in a gas concentration distribution predicted using a machine learning model with gas concentration at the reference point measured by the at least one first measurement part, the machine learning model trained to predict a gas concentration distribution with the environmental information, which includes at least one of the gas concentration, wind direction, and wind speed of a gas measured by the at least one first measurement part as an explanatory variable and a gas concentration distribution as an objective variable; tune the machine learning model such that the objective function is minimized; and estimate based on the gas concentration distribution predicted using the tuned machine learning model the gas leak position.

In any of the management devices, the environmental information may further include at least one of wind direction, wind speed, temperature and humidity, atmospheric pressure, solar radiation, and rainfall.

In any of the management devices, the at least one first measurement part may include a gas sensor that is installed within a range of a vertical deployment angle of 60 degrees with reference to a height of a position where gas leak may occur and that measures concentration of the gas as the measurement target.

In any of the management devices, a ventilation time of the at least one first measurement part may be a time until 90% of an enclosure volume of the at least one first measurement part is ventilated by diffusion ventilation.

In any of the management devices, there may be included an issue part issuing a certificate indicating an authentication result by the authentication part.

In any of the management devices, the issue part may store the certificate in a digital repository accessible and referable by a third party.

In any of the management devices, the issue part may refer to the digital repository and issue the certificate further indicating a past authentication result identified based on a past certificate of the operator related to the gas leak.

In any of the management devices, there may be included a notification part notifying the gas leak position to a predetermined notification destination.

In any of the management devices, there may be included a calibration information acquisition part acquiring calibration information including at least one of a calibration period at which calibration of the at least one first measurement part is to be executed, a calibration date and time at which calibration was executed by the at least one first measurement part, a calibration method, and calibrator information. In a case where the calibration information indicates that the at least one first measurement part is calibrated according to predetermined calibration conditions, the authentication part may authenticate the identified operator related to the gas leak as an operator that caused the gas leak.

A management system according to one aspect of the disclosure may include the management device and the at least one first measurement part.

An authentication device according to one aspect of the disclosure may include a communication interface configured to be capable of communicating with at least one first measurement part; an arithmetic device including at least one processor configured to be capable of communicating with the communication interface; and a memory device storing a predetermined authentication protocol, and the communication interface may be configured to receive environmental information, which includes concentration of at least one type of measurement target gas from the first measurement part. The arithmetic device may include an environmental information acquisition part acquiring the environmental information via the communication interface; an estimation part estimating based on the environmental information an occurrence of leak of the measurement target gas and a position where the leak occurred as a gas leak position; an operator identification part identifying, based on operator information, which includes a combination of an installation position of gas equipment and identification information of an operator managing the gas equipment as well as the gas leak position estimated by the estimation part, an operator related to the gas leak; and an authentication part authenticating based on the authentication protocol the identified operator related to the gas leak as an operator that caused the gas leak, and issuing an electronic certificate for the operator authenticated by the authentication part and transmitting the electronic certificate via the communication interface.

A management method according to one aspect of the disclosure may include: acquiring, by an environmental information acquisition part, environmental information from at least one first measurement part that measures the environmental information of a measurement target. The management method may include: estimating, by an estimation part based on the environmental information a fluid leak position indicating a position where fluid leaked. The management method may include: identifying, by an operator identification part, based on operator information indicating identification information for identifying an operator and position information related to the operator as well as the fluid leak position, an operator related to the fluid leak.

Note that the above summary of the disclosure does not enumerate all of the features of the disclosure. Moreover, sub-combinations of these feature groups may also constitute disclosures.

Hereinafter, the disclosure is described through embodiments of the disclosure, but the following embodiments do not limit the disclosure according to the claims. Moreover, not all combinations of features described in the embodiments are necessarily essential to the solution of the disclosure.

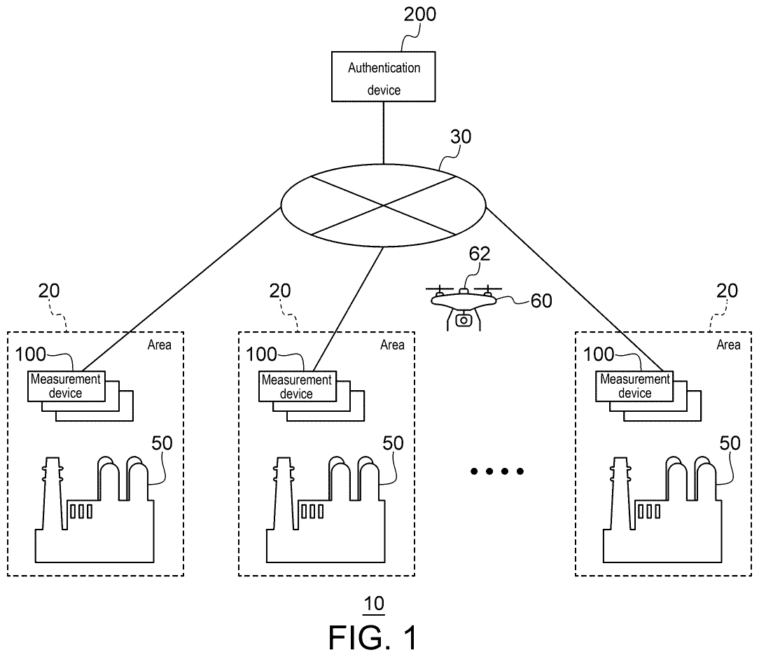

FIG. 1 is a diagram illustrating an example of an overall configuration of an authentication system 10 according to the present embodiment. The authentication system 10 includes a plurality of measurement devices 100, a UAV 60, and an authentication device 200. Each of the plurality of measurement devices 100 is disposed at equipment 50 managed by each operator or around the equipment 50. The equipment 50 may be a plant. The plant may be an industrial plant such as a chemical or bio plant, a wellhead or storage/transportation equipment (pipeline, LNG vessel, etc.) of a gas field or oil field, a plant that performs management control of the surroundings thereof, a plant that performs management control of power generation such as hydraulic, thermal, or nuclear power, a plant that performs management control of environmental power generation such as solar or wind power, a plant that performs management control of water supply and sewage or dams, a waste treatment equipment, a landfill, or the like.

The authentication device 200 may be a computer including a CPU and a memory, and may execute various functions by the CPU executing various programs stored in the memory. A measurement device 100, the UAV 60, and the authentication device 200 communicate with each other via a network 30. The authentication device 200 is an example of a management device.

Each of the plurality of measurement devices 100 is disposed in an area 20 where the equipment 50 to be monitored exists. The plurality of areas 20 may partially overlap in some regions.

The measurement device 100 measures a physical quantity related to the environment of the area 20. The measurement device 100 is fixed at a predetermined position within the area 20. The measurement device 100 may measure at least one of the following of a fluid as the measurement target: concentration, dust amount, temperature, humidity, noise, illuminance, vibration, an electromagnetic wave, an X-ray dose, a radiation dose, a wind direction, a wind speed, an atmospheric pressure, a rainfall, a solar radiation, an ozone concentration, a turbidity, a chromaticity, an oil film, or a conductivity in the area 20. Each physical quantity may be associated with a measured time and position coordinates.

Here, the fluid includes a gas, a liquid, or a mixture thereof. For example, the fluid may be a substance having fluidity such as a gas, a refrigerant, a fuel, an oil, a dust, or a pollutant that leaks into an external environment such as the atmosphere or a river. When the fluid is a gas, the measurement device 100 may measure gas concentration.

The equipment 50 provided in the area 20 includes, for example, a pipeline, and the measurement device 100 includes a gas sensor that detects a gas leak of a gas passing through the pipeline. When the measurement device 100 includes a gas sensor, gas concentration of gas as the measurement target may be measured according to a non-dispersive infrared absorption method, a Tunable Diode Laser Absorption Spectroscopy (TDLAS) method, a Differential Absorption LiDAR (DIAL) method, a Time Correlated Single Photon Counting (TCSPC) method, a photoacoustic method, a semiconductor method, a solid electrolyte method, a thermal conductivity method, an acoustic wave method, an optical gas imaging method, or a capacitance method. The gas as the measurement target may be carbon dioxide, water, or oxygen. The gas as the measurement target may be a combustible gas such as methane, propane, ethanol, hydrogen, ethylene, or MCH (methylcyclohexane). The gas as the measurement target may be a toxic gas such as carbon monoxide, hydrogen sulfide, formaldehyde, or ammonia. The gas as the measurement target may be a greenhouse gas such as carbon dioxide, nitrous oxide, or refrigerant gas.

When the measurement device 100 includes a gas sensor, at least the gas sensor may be installed within a range of a vertical deployment angle of 60 degrees based on a height of a position where a gas leak may occur, and may measure concentration of a gas as the measurement target. The position where a gas leak may occur includes, for example, flange, joint, welded portion, curved pipe portion, flexible piping, valve (such as safety valve, air valve, and check valve), gasket, and compressor. Moreover, at least the gas sensor may measure concentration of a gas on a downwind side based on a height of a position where a gas leak may occur. For example, when the equipment 50 includes a cooling fan, the gas sensor may be disposed on a downwind side of the fan. When a ventilation time of the gas sensor is defined as a time until 90% of an enclosure volume of the gas sensor is ventilated by diffusion ventilation, the ventilation time may be 60 seconds or less. Similarly, a 90% response time of the gas sensor may be 60 seconds or less. For example, when the ventilation time of the gas sensor is within 60 seconds, a gas remaining inside an enclosure of the gas sensor is rapidly discharged, such that a response delay to a variation in gas concentration outside the enclosure of the gas sensor may be reduced, and a gas leak position can be estimated more accurately.

The height of the position where a gas leak may occur may be any height within the range of the maximum height and the minimum height of gas piping or gas processing equipment of the equipment that processes the gas as the measurement target.

The vertical deployment angle may be a full angle of a fan shape extending upward and downward from a reference line, where the reference line is a horizontal line passing through the height of the position where a gas leak may occur and parallel to the ground surface. For example, a vertical deployment angle of 60 degrees may mean that the full angle of the fan shape is 60 degrees, and the half angle with respect to the reference line is 30 degrees upward and downward.

When the measurement device 100 includes a gas sensor, at least the gas sensor is installed on the ground around a position where a gas leak may occur, and may measure the concentration of the gas as the measurement target. Furthermore, when the gas sensor has explosion-proof capacities, it may be arranged in an explosion-proof area near the position where a gas leak may occur. Here, the explosion-proof area may be a space where flammable gas, vapor, or dust exists and where there is a possibility of explosion or fire due to an ignition source such as sparks or high temperature.

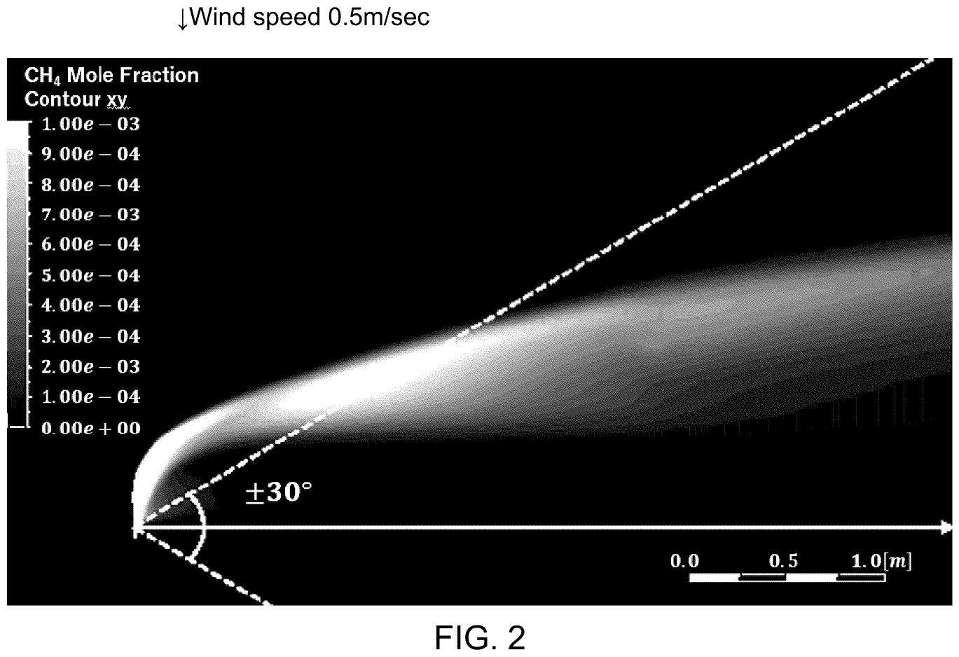

FIG. 2 is a diagram illustrating an example of a simulation in which gas concentration distribution of gas diffusion in the downwind direction when a gas leak occurs is numerically calculated. Here, the analysis conditions for the simulation are leaked gas type: methane, gas concentration: 4%, leak amount: 100 L/min, ejection hole diameter: ½ inch, and wind speed: 0.5 m/sec. For example, in a case where a gas sensor is installed within a range of a vertical deployment angle of 60 degrees based on the height at which gas leak occurs, the gas concentration measured by the gas sensor becomes higher, such that the gas leak position can be estimated more accurately.

For the same reason, the gas sensor is preferably an optical gas sensor with a fast response speed using a principle such as a non-dispersive infrared absorption method, a tunable diode laser absorption spectroscopy method, or a photoacoustic method. Moreover, for the same reason, when an enclosure volume of the gas sensor is V, a ventilation opening area is S, a flow velocity is u, and a ventilation time is t, the ventilation time t may be determined by “t=V/(S·u)”, and for example, a ratio “V/S” of the ventilation volume V to the ventilation opening area S is preferably 200 or less.

Moreover, for the same reason, it is preferable that ventilation openings are installed on each of an upstream side and a downstream side with respect to a flow field of the gas, such that the gas flows rapidly inside the enclosure and the gas inside the enclosure is replaced more quickly.

Moreover, for the same reason, it is desirable that the gas sensor includes a fan for ventilation. Moreover, the gas sensor is preferably small. For example, when the gas sensor is small, the ventilation volume V becomes small, such that the ventilation time t may be reduced. Moreover, when the gas sensor is small, an influence of the gas sensor becoming an obstacle and disturbing a flow field becomes smaller, such that a gas leak position may be estimated more accurately. For example, the flow field may be a flow field of a gas that varies depending on wind direction and wind speed.

The measurement device 100 may include a dust sensor, a temperature and humidity sensor, a noise sensor, an illuminance sensor, a vibration sensor, an electromagnetic wave measuring instrument, an X-ray measuring instrument, a radiation measuring instrument, a LiDAR (Light Detection And Ranging), a radar, a visible light imaging device, an infrared light imaging device, an atmospheric pressure sensor, or an airflow inspector. Moreover, the measurement device 100 may include a clock and a position sensor.

An unmanned aerial vehicle (UAV) 60 is an example of a mobile object. The mobile object is a concept including an artificial satellite, such as a flying object that moves in the air, a vehicle or a multi-legged walking machine that moves on the ground, and a ship that moves on the water. The flying object that moves in the air is a concept including, in addition to the UAV, other aircraft, airships, helicopters, and the like that move in the air. The UAV 60 includes a measurement device 62. The measurement device 62 is an example of a mobile second measurement part. A function of the measurement device 62 may be the same as that of the measurement device 100. The measurement device 62 may include the same sensor as the measurement device 100. That is, the UAV 60 may be equipped with the same sensor as the measurement device 100 as the measurement device 62. Moreover, the unmanned aerial vehicle (UAV) 60 may include a clock and a position sensor. Alternatively, the mobile object may be a measurement device 62 including a position sensor and may be portable. For example, the mobile object may be a portable measurement device with GPS carried by a worker.

The authentication device 200 collects environmental information based on measurement data measured by the plurality of measurement devices 100 and the measurement device 62 of the UAV 60.

In recent years, as a measure against climate change, MMRV (Measurement, Monitoring, Reporting, and Verification) for greenhouse gas leaks has been mandated for energy-related operators, and penalty fees are incurred according to the gas leak volume. When imposing sanctions such as penalty fees in this manner, it is preferable to be able to objectively prove the fact that an operator caused a gas leak. For example, by objectively authenticating that an operator caused a gas leak, the fact that the operator caused a gas leak can be objectively proved.

Therefore, in the authentication system 10 according to the present embodiment, when it is detected that a gas leak is occurring based on the environmental information from the measurement device 100, which operator caused the gas leak is identified, and the result of the identification is authenticated. Furthermore, in the authentication system 10 according to the present embodiment, an electronic certificate indicating that the operator caused the gas leak is issued. Thereby, the fact that the operator caused the gas leak can be objectively authenticated.

Here, the authentication may be a process of confirming that the identification result is derived through a legitimate procedure and has not been tampered with, and guaranteeing the reliability and authenticity of the identification result. Moreover, in order to ensure the reliability and authenticity of the identification result, it is preferable that the authentication is executed by a third-party organization, a neutral authentication entity, a public institution, or a system under the jurisdiction of these, which is not the operator subject to the identification.

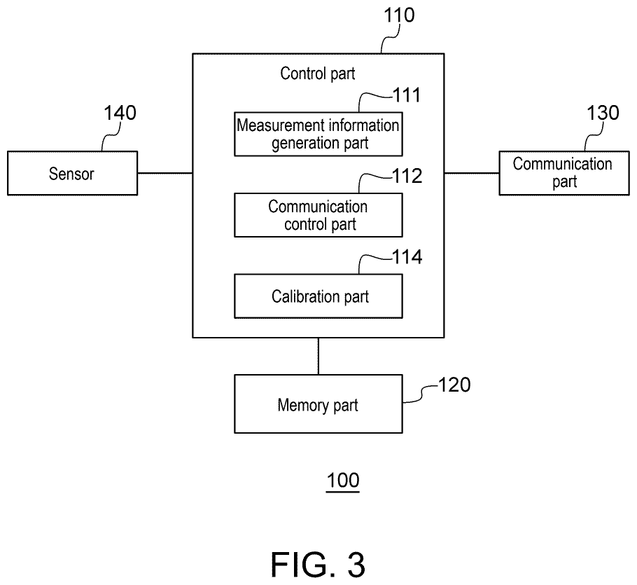

FIG. 3 is a diagram illustrating an example of functional blocks of the measurement device 100. The measurement device 100 includes a control part 110, a memory part 120, a communication part 130, and a sensor 140.

The control part 110 may be configured by a microprocessor such as a CPU or MPU, a microcontroller such as an MCU, or the like. The sensor 140 measures a physical quantity related to the environment in the area 20. The memory part 120 stores a program or the like for realizing processing executed by the control part 110 for the measurement device 100 to measure a physical quantity and transmit environmental information indicating the measured physical quantity to the authentication device 200. The memory part 120 stores measurement data measured by the sensor 140 or environmental information based on the measurement data. The communication part 130 includes a communication interface and communicates with the authentication device 200 via the network 30.

The sensor 140 measures a physical quantity related to the environment in the area 20 at predetermined intervals, for example, at intervals of once or more per 60 seconds. The sensor 140 may include a light emitting part that emits infrared light and a light receiving part that receives infrared light transmitted through the gas as the measurement target, and may measure the gas concentration of the gas by utilizing the infrared absorption characteristics of the gas as the measurement target. The sensor 140 may measure the gas concentration of the gas in the area 20 as a physical quantity related to the environment. The sensor 140 may measure measurement data indicating the gas concentration of the gas leaked from the pipeline in the area 20. The measurement device 100 may have a plurality of sensors 140 that measure a plurality of types of physical quantities. The measurement device 100 may have a plurality of sensors 140 that measure at least one of wind direction, wind speed, temperature and humidity, atmospheric pressure, solar radiation, and rainfall.

The control part 110 includes a measurement information generation part 111 and a communication control part 112. The measurement information generation part 111 may generate environmental information from the measurement data measured by the sensor 140. The environmental information may include the gas concentration of the gas. The environmental information may further include at least one of wind direction, wind speed, temperature and humidity, atmospheric pressure, solar radiation, and rainfall. The measurement information generation part 111 may generate environmental information by statistically processing the measurement data measured by the sensor 140. The measurement information generation part 111 may generate statistical information including at least one of an average value, a maximum value, a minimum value, a variance, a moment, and a histogram from the measurement data as the environmental information.

The communication control part 112 transmits the environmental information to the authentication device 200 by controlling the communication part 130. A calibration part 114 executes calibration of the measurement device 100. The characteristics of the sensor 140 may change over time. The characteristics of the sensor 140 refer to, for example, characteristics of an optical element or the like in the case where the sensor 140 is an optical element and is a gas sensor such as a CO₂ (carbon dioxide) sensor that follows a non-dispersive infrared absorption method for measuring gas concentration by infrared light. The characteristics of the optical element or the like may change over time. For this reason, the measurement device 100 executes calibration for correcting measurement accuracy.

The calibration part 114 may execute calibration based on the gas concentration calculated by itself and a predetermined reference gas concentration in the area 20. The calibration part 114 may execute calibration by correcting a reference value (baseline value) of the gas concentration based on correction information provided from the authentication device 200 described later. The calibration part 114 may correct a coefficient for calculating the gas concentration such that the gas concentration calculated by itself matches the reference gas concentration when a condition that the gas concentration in the area 20 becomes the reference gas concentration is satisfied. The measurement device 100 may correct the coefficient such that the minimum value of the gas concentration calculated by itself within a predetermined period matches the gas concentration of the reference value. The predetermined period may be a period in which the gas concentration of the target gas is most likely to be the lowest. For example, it may be a period during which operation of a device that may generate gas is stopped.

The memory part 120 may store calibration information including at least one of a calibration period at which calibration is to be executed by the calibration part 114, a calibration date and time executed by the calibration part 114, a calibration method, and calibrator information. The calibration method is a method of calibration executed by the calibration part 114. The calibration method may indicate, for example, that calibration is executed during a period in which the gas concentration of the target gas is most likely to be the lowest. The calibration method may indicate, for example, a period considering weather or the like during which measurement of gas concentration by the measurement device 100 may be performed stably.

The communication control part 112 may transmit the calibration information together with the environmental information to the authentication device 200. The calibration method may indicate at least one of a type of gas as the measurement target, concentration, number of concentration points, traceability system, accuracy of concentration, gas components, gas purchase date/calibration certificate issue date, gas vendor, gas purchaser, container symbol number, gas expiration date, type of adjustment parameters (for example, zero, span, offset, sensitivity), environmental information at the time of calibration (temperature, humidity, atmospheric pressure, and date and time), and residual pressure of calibration gas.

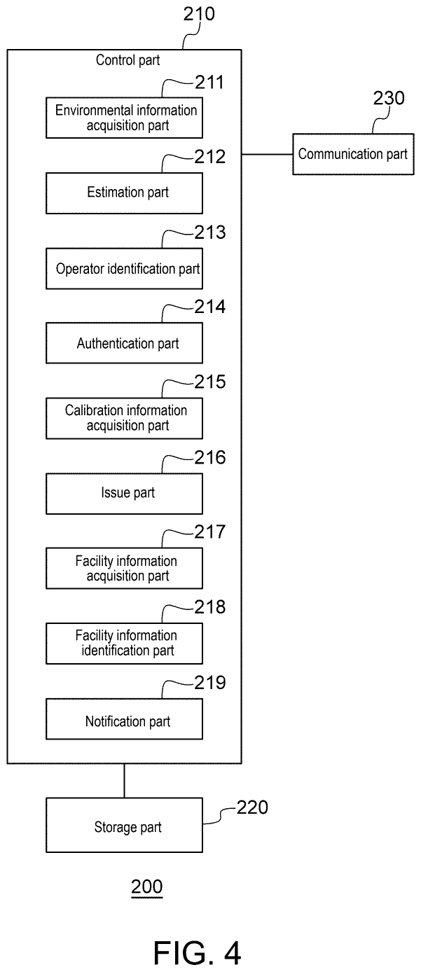

FIG. 4 is a diagram illustrating an example of functional blocks of the authentication device 200. The authentication device 200 includes a control part 210, a storage part 220, and a communication part 230. The control part 210 may be configured by a microprocessor such as a CPU or MPU, a microcontroller such as an MCU, or the like. The storage part 220 is a database that stores environmental information indicating various physical quantities related to the environment collected from each measurement device 100 and the measurement device 62. Moreover, the storage part 220 stores an authentication protocol defined by an authentication organization that authenticates gas emission volume. The communication part 230 includes a communication interface and communicates with the measurement device 100 via the network 30. Moreover, the communication interface included in the communication part 230 is configured to be capable of communicating with at least one arithmetic device constituting the control part 210.

The control part 210 includes an environmental information acquisition part 211, an estimation part 212, an operator identification part 213, an authentication part 214, a calibration information acquisition part 215, an issue part 216, a facility information acquisition part 217, a facility information identification part 218, and a notification part 219.

The environmental information acquisition part 211 acquires environmental information from at least one measurement device 100 that measures environmental information including at least gas concentration of the gas as the measurement target. The environmental information acquisition part 211 may acquire environmental information from each of a plurality of measurement devices 100 provided in each area 20.

The estimation part 212 estimates a gas leak position indicating a position where gas leaked based on the environmental information. The estimation of the gas leak position includes detection that gas leak has occurred. The estimation part 212 may estimate the gas leak position using an inverse analysis model, a statistical model, an atmospheric dispersion prediction model, or a machine learning model indicating a relationship between the environmental information and the gas leak position.

The estimation part 212 derives a gas concentration distribution based on environmental information including at least one of, for example, a position of a measurement point of gas released into the atmosphere and diffused, gas concentration of the gas, and wind direction and wind speed. The estimation part 212 assumes gas release from one gas leak position and derives a gas concentration distribution corresponding to the wind direction and wind speed from that gas leak position according to a calculation model such as a plume model. The estimation part 212 estimates the gas leak position based on the gas concentration distribution derived according to the calculation model and the gas concentration distribution derived based on the environmental information. The estimation part 212 may estimate, as the gas leak position, a gas leak position assumed in a gas concentration distribution that is most similar to the gas concentration distribution derived based on the environmental information, among the gas concentration distributions derived according to the calculation model.

The estimation part 212 may estimate a gas leak position based on the environmental information acquired from the measurement device 100, using a trained machine learning model that uses environmental information including at least one of gas concentration of the gas, wind direction and wind speed as an explanatory variable and uses the gas leak position as an objective variable. The estimation part 212 may perform machine learning according to a supervised learning algorithm using training data that uses environmental information including at least one of gas concentration of the gas, wind direction and wind speed as an explanatory variable and uses the gas leak position as an objective variable, thereby generating a trained machine learning model that estimates a gas leak position from the environmental information, and may store the trained machine learning model in the storage part 220. The algorithm may be an algorithm of any method such as a neural network, a support vector machine, a plurality of regression analysis, or a decision tree.

The estimation part 212 may estimate the gas leak position by calculating behavior of particles by tracing back in time using a particle method based on environmental information including at least one of gas concentration of the gas measured by at least one first measurement part, wind direction and wind speed. The estimation part 212 may generate a flow field and particles around the first measurement part based on the environmental information, estimate past positions of the particles by tracing back in time, and estimate the gas leak position. For example, the particles may be gas molecules of the gas as the measurement target. For example, the flow field may be a flow field of gas that varies depending on wind direction and wind speed.

The estimation part 212 may train a machine learning model that predicts gas concentration distribution using training data that uses environmental information including at least one of gas concentration of the gas, wind direction and wind speed measured over a certain period as an explanatory variable and uses gas concentration distribution as an objective variable, compare gas concentration at a reference point in the gas concentration distribution predicted using the trained machine learning model with actual gas concentration at the reference point measured by the sensor 140 to define an objective function, tune the machine learning model such that the objective function becomes optimal, and estimate the gas leak position based on the gas concentration distribution predicted using the tuned machine learning model.

For example, the machine learning model may be a Gaussian plume model, a puff model, a Lagrangian model, or a Lorentz model. For example, the objective function may be any method such as mean squared error, relative error, log-likelihood, clustering index, or cross-entropy method.

For example, the estimation of the gas leak position may be any method such as least squares method, gradient descent method, Bayesian estimation method, Markov chain Monte Carlo method, gradient boosting, random forest, Kalman filter, or genetic algorithm.

The estimation part 212 may generate a trained machine learning model that identifies the gas leak position from environmental information, using environmental information including at least one of past gas concentration of the gas, wind direction and wind speed, and the gas leak position identified in the past as training data. The estimation part 212 may correct the gas concentration based on environmental information including at least one of gas concentration, wind direction, and wind speed of the plurality of measurement devices 100. The estimation part 212 may generate a machine learning model that estimates the gas leak value excluding environmental information below a predetermined threshold, and estimate the gas leak position.

The operator identification part 213 identifies an operator related to the gas leak based on operator information indicating identification information for identifying an operator and position information related to the operator, and the gas leak position estimated by the estimation part 212. The identification information may include an operator name, an operator code, and the like. The identification information may include information related to management, operation, or ownership of gas equipment as information for identifying the operator. Moreover, the identification information may include contact information linked to the operator name or the operator code. The position information may be ID information of equipment assigned to gas-related equipment (equipment 50), an operator code associated with the ID information of each equipment, and information indicating a defined area including the equipment 50. The information indicating the defined area including the equipment 50 included in the position information may be, for example, information such as an address indicating a specific area on a map. The information indicating the defined area including the equipment 50 included in the position information may be latitude and longitude information of the defined area including the equipment 50. The position information may further include building height information of the equipment 50. The position information may be land, facility, or equipment position related to gas-related equipment and an operator name or operator code registered as a manager, operator, or owner thereof, or registration information in which these are described. The operator information may further include information related to inspection or maintenance of land, facility, or equipment. The operator identification part 213 may identify an operator that manages, operates, or owns the equipment 50 for which the distance between the position identified by the position information and the gas leak position estimated by the estimation part 212 satisfies a predetermined distance range, as an operator related to the gas leak. The operator identification part 213 may identify an operator that manages, operates, or owns the equipment 50 for which the distance between the position identified by the position information and the gas leak position estimated by the estimation part 212 is shorter than a predetermined distance, as an operator related to the gas leak.

Here, the gas leak position estimated based on environmental information acquired from a plurality of measurement devices 100 fixed at predetermined positions indicates an area having a certain range. Such an area may overlap with a plurality of areas 20 of a plurality of operators. For example, in a case where a plurality of equipment 50 of a plurality of operators is densely provided in a complex or the like, it may not be possible to narrow down the operator that caused the gas leak from among the plurality of operators based on the estimated gas leak position.

Therefore, in a case where the operator identification part 213 identifies a plurality of operators related to the gas leak based on the operator information and the gas leak position, the environmental information acquisition part 211 may further acquire environmental information including the gas concentration of gas around the gas leak position from the measurement device 62 mounted on at least one UAV 60 as additional environmental information. The operator identification part 213 may identify the operator related to the gas leak from among the plurality of operators further based on the additional environmental information.

The UAV 60 moves to the vicinity of the estimated gas leak position in response to a request from the authentication device 200, and the measurement device 62 measures the physical quantity related to the environment around the estimated gas leak position.

The UAV 60 transmits additional environmental information obtained by measurement by the measurement device 62 to the authentication device 200. The operator identification part 213 further narrows down the estimated gas leak position based on the additional environmental information. The estimation part 212 may re-estimate a second range, which is a further reduction of the first range of the gas leak position estimated based on the environmental information of the measurement device 100, as the range of the gas leak position based on the additional environmental information. The estimation part 212 may derive a gas concentration distribution based on the additional environmental information, and re-estimate a second range, which is a further reduction of the first range of the gas leak position estimated based on the environmental information of the measurement device 100 from the gas concentration distribution, as the range of the gas leak position.

In a case where the second range is estimated as a gas leak position overlapping with the first range, the additional environmental information and the second range may be transmitted to the authentication device 200 or notified to the operator as having a high possibility of gas leak. For example, based on the gas concentration, wind direction, and wind speed information of gas from a plurality of measurement devices 100, the first range may be estimated assuming that the gas leak position is within a cone apex angle of 120° on the upwind side when the gas is detected. There may be a plurality of first ranges that are gas leak position candidates.

The UAV 60 may determine the range and route for acquiring environmental information by the measurement device 62 based on the environmental information acquired from the measurement device 100 and the environmental information acquired from the measurement device 62. For example, the route of the UAV 60 may scan a conical region including the first range in a zigzag pattern, starting from a point showing the highest gas concentration value among the plurality of measurement devices 100, or from an arbitrary point on the downwind side from the first range. A machine learning model may be trained and generated based on the environmental information acquired from the measurement device 62 of the UAV 60.

The first range of the gas leak position estimated by the estimation part 212 is estimated based on the environmental information including at least one of gas concentration, wind direction, and wind speed of gas acquired by the measurement device 62. Based on the environmental information of the measurement device 100 inside and around the first range, a second range that is a further reduction of the first range of the estimated gas leak position may be re-estimated as the range of the gas leak position. The estimation part 212 may derive a gas concentration distribution based on the additional environmental information, and re-estimate a second range, which is a further reduction of the first range of the gas leak position estimated based on the environmental information of the measurement device 62 from the gas concentration distribution, as the range of the gas leak position.

The operator identification part 213 may identify an operator related to the gas leak from among a plurality of operators based on the second range. The operator identification part 213 may identify from among a plurality of operators an operator managing the equipment 50 included in the second range as an operator related to the gas leak.

Furthermore, in a case where the estimation part 212 estimates that the gas leak position estimated based on the environmental information of the measurement device 100 from the gas concentration distribution to be a position of a facility or equipment that has not been inspected or maintained for a sufficient period or frequency, or for required items, based on the operator information, the operator identification part 213 may estimate that the gas leak is occurring due to human factors or negligence.

Furthermore, in a case where the gas leak position estimated based on the environmental information of the measurement device 100 from the gas concentration distribution is estimated to be a position of land or a facility that does not belong to any operator, the operator identification part 213 may identify or estimate that the gas leak is occurring due to natural factors rather than human factors.

Leak information related to an operator identified by the operator identification part 213 as having caused a gas leak may be retained in a form that prevents unauthorized alteration, may be encrypted, and may be encrypted based on distributed ledger technology.

The authentication part 214 authenticates the identified operator related to the gas leak as an operator that caused the gas leak. The authentication part 214 authenticates according to an authentication protocol stored in the storage part 220. The calibration information acquisition part 215 acquires calibration information including at least one of a calibration period at which calibration of the measurement device 100 is to be executed, a calibration date and time at which calibration was executed by the measurement device 100, a calibration method, and calibrator information. The authentication part 214 may authenticate the identified operator related to the gas leak as an operator that caused the gas leak in a case where the calibration information indicates that the measurement device 100 has been calibrated according to predetermined calibration conditions. In a case where the operator identification part 213 identifies a plurality of operators for which a distance between a position identified from the position information and the gas leak position satisfies a predetermined distance range, the authentication part 214 may authenticate that each of the plurality of operators caused the gas leak at respective ratios identified according to a magnitude of a distance between positions of the plurality of operators and the gas leak position. The authentication part 214 may determine a gas leak volume of each of the plurality of operators such that the gas leak volume becomes larger as a distance between a position of an operator and the gas leak position is shorter.

For example, the authentication part 214 may identify a calibration date and time of the measurement device 100 by referring to the calibration information, and may authenticate the operator if the calibration date and time is within a predetermined period from the present (for example, within one month). Alternatively, the authentication part 214 may identify a calibration method of the measurement device 100 by referring to the calibration information, and may authenticate the operator in a case where the calibration method satisfies a predetermined reference method.

The issue part 216 issues an electronic certificate certifying that the operator authenticated by the authentication part 214 is an operator that caused the gas leak. The electronic certificate is an electronic document certifying that the operator caused the gas leak, to which an electronic signature of an approval authority is attached. The communication part 230 identifies a predetermined destination such as an administrator of the equipment 50 that caused the gas leak based on the identification information, and notifies the gas leak position together with the electronic certificate. In a case where the operator authenticated by the authentication part 214 has not caused a gas leak, the issue part 216 may issue an electronic certificate certifying that the corresponding operator is an excellent operator. The electronic certificate may indicate at least one of an operator name, an operator number, a gas leak detection date and time, a gas leak report date and time, a certificate issue date and time, a sensor identification number, a sensor model number, a sensor manufacturer name, sensor calibration information, a certificate number, gas leak position information, a gas leak volume, a reliability of a gas leak estimation result, equipment information, a degree of excellence of the operator, a certificate issuer name, and an electronic signature of the certificate issuer.

The authentication device 200 may store at least one of the authentication result and the electronic certificate in a repository that is also accessible to third parties. The issue part 216 may refer to a digital repository and issue a certificate further indicating past authentication results identified based on past certificates of an operator related to the gas leak. For example, in a case where a third party is a gas consumer operator, the gas consumer operator may select an excellent gas sales operator based on the authentication result stored in the repository. Additionally, the authentication system may issue a credit or a penalty based on a difference between an amount of gas leaked from an operator authenticated by the authentication part 214 during a certain period and a reference value. The credit is, for example, a carbon credit. For example, the penalty is a WEC (Waste Emission Charge).

The facility information acquisition part 217 acquires equipment information corresponding to the gas leak position by referring to equipment information indicating information about the equipment 50 that processes the gas as the measurement target and the position of the equipment 50. The equipment information may indicate at least one of a type of the equipment 50, a type of a device provided in the equipment 50, a type of a component provided in the equipment 50, operating conditions of the equipment 50, and a welded part. The device provided in the equipment 50 may be at least one of a flare stack, an exhaust port, a compressor, a heat exchanger, a tank, a well, a separator, a dehydrator, a pipeline, a flow meter, and a pressure gauge. The type of component provided in the equipment 50 may be, for example, a cylinder, a flange, a flexible pipe, or a valve (a safety valve, an air valve, a check valve, or the like). The type of device and component may include at least one of a manufacturer, a model, and a date of manufacture. The operating conditions of the equipment 50 indicate operation content of a device to be performed to execute predetermined processing to be performed by the equipment 50, setting content of the device, a type of gas to be handled, a gas production volume, a gas consumption volume, a gas transportation volume, a gas storage volume, a temporal change in the gas storage volume, an operating time, an energy consumption volume, an in-house power generation volume, a purchased power volume, an inspection record, a repair record, a greenhouse gas emission volume, and the like. For example, environmental information acquired when gas is intentionally released by a flare stack may not be used for estimation of the gas leak position because the intentional gas release is misidentified as a gas leak, that is, the gas leak position may be estimated by selecting environmental information based on intentional gas release information as equipment information. For example, since a welded part is prone to developing pinholes due to aging deterioration and causing gas leaks, the gas leak position may be estimated with high accuracy by using the equipment information.

For example, when respective equipment 50 that operates under the same operating conditions and includes the same type of device and component operates in the same environment, an abnormality may occur in the equipment 50 at similar timing, and a gas leak may occur at similar timing.

Therefore, the facility information identification part 218 refers to the equipment information and identifies equipment information of at least one other equipment 50 related to the equipment information corresponding to the gas leak position. The facility information identification part 218 may refer to the equipment information and identify at least one other equipment 50 that operates under the same operating conditions as the equipment 50 corresponding to the gas leak position and includes the same type of device and component, by comparing the equipment information of the equipment 50 corresponding to the gas leak position with the equipment information of the other equipment 50. The facility information identification part 218 does not necessarily need to identify a specific leak site, and may identify the equipment having the leak site. For example, when a gas leak has occurred from a specific valve of a certain gas extraction equipment, the facility information identification part 218 does not necessarily need to identify the valve where the gas leak is occurring, and may identify the gas extraction equipment having the corresponding valve.

The environmental information acquisition part 211 acquires environmental information around the position of at least one other equipment 50 from the measurement device 100 provided in the area 20 where the at least one other equipment 50 exists.

The estimation part 212 estimates, based on past environmental information around the gas leak position and environmental information around the position of at least one other equipment 50, other equipment 50 that may cause a gas leak within a predetermined period among the at least one other equipment 50. The past environmental information may be environmental information measured by the measurement device 100 within a predetermined period up to the time when the gas leak was detected. The predetermined period up to the time when the gas leak was detected may be one hour, one day, one week, one month, or the like. When the similarity between the environmental information of the area 20 where at least one other equipment 50 exists and the past environmental information around the gas leak position is equal to or greater than a threshold, the estimation part 212 may estimate the other equipment 50 as other equipment 50 that may cause a gas leak within a predetermined period.

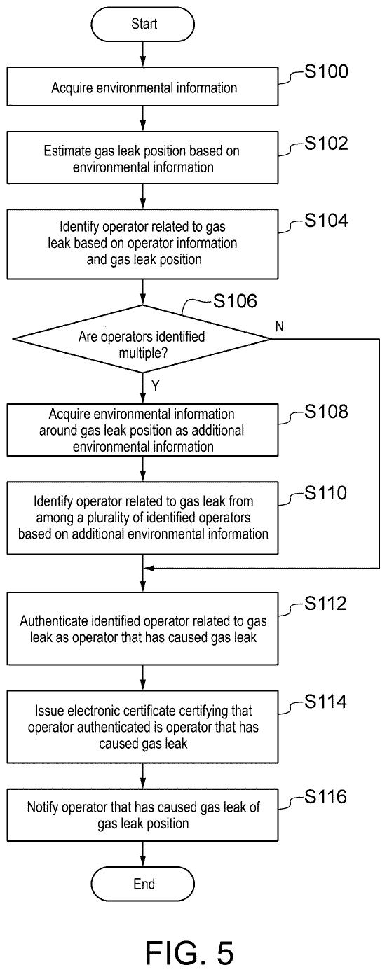

FIG. 5 is a flowchart illustrating an example of a procedure for authenticating an operator that caused a gas leak.

The environmental information acquisition part 211 acquires environmental information from the measurement device 100 provided in each of the plurality of equipment 50 (S100). The estimation part 212 estimates a gas leak position indicating a position where a gas leak has occurred based on the environmental information (S102).

The operator identification part 213 identifies an operator related to the gas leak based on operator information indicating identification information for identifying an operator and position information related to the operator, and the gas leak position estimated by the estimation part 212 (S104).

The environmental information acquisition part 211 determines whether the operators identified by the operator identification part 213 are multiple (S106). In the case of a plurality of operators, the environmental information acquisition part 211 acquires environmental information including gas concentration of gas around the gas leak position from the measurement device 62 mounted on at least one UAV 60 as additional environmental information (S108).

The operator identification part 213 identifies an operator related to the gas leak from among the plurality of operators further based on the additional environmental information (S110). The estimation part 212 derives a gas concentration distribution based on the additional environmental information, and estimates, as a range of the gas leak position, a second range that is further narrowed from a first range of the gas leak position estimated based on the environmental information of the measurement device 100 from the gas concentration distribution. The operator identification part 213 identifies, as an operator related to the gas leak, an operator that manages equipment 50 included in the second range from among the plurality of operators.

In the case of one operator being identified in step S104, or in the case of one operator being identified from among the plurality of operators in step S110, the authentication part 214 authenticates the identified operator related to the gas leak as an operator that caused the gas leak (S112). Here, the calibration information acquisition part 215 acquires calibration information, and the authentication part 214 may authenticate the identified operator related to the gas leak as an operator that caused the gas leak in the case of the calibration information indicating that the measurement device 100 has been calibrated according to predetermined calibration conditions.

The issue part 216 issues an electronic certificate certifying that the operator authenticated by the authentication part 214 is an operator that caused the gas leak (S114). The communication part 230 notifies the operator that manages the equipment 50 that caused the gas leak of the gas leak position together with the electronic certificate (S116).

As described above, according to the authentication system 10 according to the present embodiment, in the case of a gas leak occurring, it is possible to identify which operator has caused the gas leak and authenticate the result of the identification. Moreover, an electronic certificate indicating that the operator has been authenticated as an operator that caused the gas leak may also be issued. This enables objective authentication of the fact that the operator has caused the gas leak.

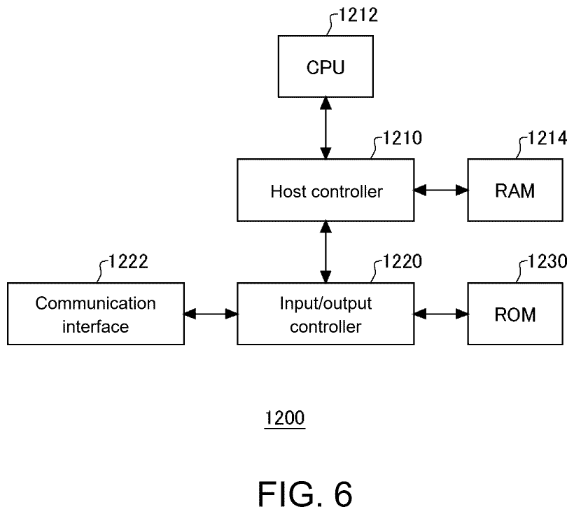

FIG. 6 shows an example of a computer 1200 that may embody aspects of the present embodiment in whole or in part. A program installed in the computer 1200 may cause the computer 1200 to function as operations associated with a device according to an embodiment of the disclosure or as one or more “parts” of the device. Alternatively, the program may cause the computer 1200 to execute the operations or the one or more “parts”. The program may cause the computer 1200 to execute a process according to an embodiment of the disclosure or steps of the process. Such a program may be executed by a CPU 1212 to cause the computer 1200 to execute specific operations associated with some or all of the blocks in the flowcharts and block diagrams described herein.

The computer 1200 according to the present embodiment includes a CPU 1212 and a RAM 1214, which are interconnected by a host controller 1210. The computer 1200 also includes a communication interface 1222 and an input/output unit, which are connected to the host controller 1210 via an input/output controller 1220. The computer 1200 also includes a ROM 1230. The CPU 1212 operates according to programs stored in the ROM 1230 and the RAM 1214, thereby controlling each unit.

The communication interface 1222 communicates with other electronic devices via a network. A hard disk drive may store programs and data used by the CPU 1212 in the computer 1200. The ROM 1230 stores therein a boot program or the like executed by the computer 1200 upon activation, and/or programs dependent on the hardware of the computer 1200. Programs are provided via a computer-readable storage medium such as a CD-ROM, a USB memory, or an IC card, or via a network. The programs are installed in the RAM 1214, which is also an example of a computer-readable storage medium, or the ROM 1230, and are executed by the CPU 1212. Information processing described in these programs is read by the computer 1200 and brings about cooperation between the programs and the various types of hardware resources described above. A device or method may be configured by realizing an operation or processing of information in accordance with the use of the computer 1200.

For example, in the case of communication being executed between the computer 1200 and an external device, the CPU 1212 may execute a communication program loaded into the RAM 1214 and instruct the communication interface 1222 to perform communication processing based on processing described in the communication program. Under the control of the CPU 1212, the communication interface 1222 reads transmission data stored in a transmission buffer area provided in the RAM 1214 or a storage medium such as a USB memory, transmits the read transmission data to a network, or writes reception data received from a network to a reception buffer area or the like provided on a storage medium.

Moreover, the CPU 1212 may cause all or a portion of a file or database stored in an external storage medium such as a USB memory or the like to be read into the RAM 1214, and may execute various types of processing on the data on the RAM 1214. The CPU 1212 may then write back the processed data to the external storage medium.

Various types of information such as various types of programs, data, tables, and databases may be stored in a storage medium and subjected to information processing. The CPU 1212 may execute various types of processing on data read from the RAM 1214, including various types of operations, information processing, condition determination, conditional branching, unconditional branching, search/replacement of information, and the like, described throughout the disclosure and specified by an instruction sequence of a program, and write back the results to the RAM 1214. Moreover, the CPU 1212 may search for information in a file, a database, or the like in a storage medium. For example, in the case of a plurality of entries each having an attribute value of a first attribute associated with an attribute value of a second attribute being stored in a storage medium, the CPU 1212 may search for an entry matching a condition in which an attribute value of the first attribute is specified from among the plurality of entries, read an attribute value of the second attribute stored in the entry, and thereby obtain an attribute value of the second attribute associated with the first attribute satisfying a predetermined condition.

The program or software module described above may be stored in a computer-readable storage medium on the computer 1200 or near the computer 1200. Moreover, a storage medium such as a hard disk or RAM provided in a server system connected to a dedicated communication network or the Internet may be used as a computer-readable storage medium, thereby providing a program to the computer 1200 via a network.

A computer-readable medium may include any tangible device capable of storing instructions executed by an appropriate device. As a result, a computer-readable medium having instructions stored thereon includes an article of manufacture including instructions that may be executed to create means for performing operations specified in flowcharts or block diagrams. Examples of computer-readable media may include electronic storage media, magnetic storage media, optical storage media, electromagnetic storage media, semiconductor storage media, and the like. More specific examples of computer-readable media may include floppy (registered trademark) disks, diskettes, hard disks, random access memory (RAM), read-only memory (ROM), erasable programmable read-only memory (EPROM or flash memory), electrically erasable programmable read-only memory (EEPROM (registered trademark)), static random access memory (SRAM), compact disc read-only memory (CD-ROM), digital versatile disc (DVD), Blu-ray (RTM) disc, memory stick, integrated circuit card, and the like.

Computer-readable instructions may include either source code or object code written in any combination of one or more programming languages. The source code or object code includes conventional procedural programming languages. Conventional procedural programming languages may be assembler instructions, instruction set architecture (ISA) instructions, machine instructions, machine-dependent instructions, microcode, firmware instructions, state setting data, or object-oriented programming languages such as Smalltalk (registered trademark), JAVA (registered trademark), C++, and the like, and “C” programming language or similar programming languages. Computer-readable instructions may be provided to a processor or programmable circuit of a general-purpose computer, special-purpose computer, or other programmable data processing device locally or via a wide area network (WAN) such as a local area network (LAN), the Internet, or the like. The processor or programmable circuit may execute computer-readable instructions to create means for performing operations specified in flowcharts or block diagrams. Examples of processors include computer processors, processing units, microprocessors, digital signal processors, controllers, microcontrollers, and the like.

Although the disclosure has been described above using the embodiments, the technical scope of the disclosure is not limited to the scope described in the above embodiments. It is apparent to those skilled in the art that various modifications or improvements may be added to the above embodiments. It is apparent from the description of the claims that forms with such modifications or improvements may also be included in the technical scope of the disclosure.

It should be noted that the execution order of each process such as operations, procedures, steps, and stages in the devices, systems, programs, and methods shown in the claims, specification, and drawings may be realized in any order unless specifically indicated as “before” or “prior to,” and unless the output of a previous process is used in a subsequent process. Even if the operation flows in the claims, specification, and drawings are described using “first,” “next,” and the like for convenience, this does not mean that implementation in this order is mandatory.

What is claimed is:

1. A management device, comprising:
an environmental information acquisition part acquiring environmental information from at least one first measurement part that measures the environmental information of a measurement target;
an estimation part estimating based on the environmental information a fluid leak position indicating a position where fluid leaked; and
an operator identification part identifying, based on operator information indicating identification information for identifying an operator and position information related to the operator as well as the fluid leak position, an operator related to the fluid leak.

2. The management device according to claim 1, wherein the environmental information includes gas concentration, and the fluid is gas.

3. The management device according to claim 2, further comprising:
an authentication part authenticating the identified operator related to gas leak, which is the fluid leak, as an operator that caused the gas leak,
the management device functioning as an authentication device.

4. The management device according to claim 3,
wherein in a case where the operator identification part identifies based on the operator information and the gas leak position a plurality of operators related to the gas leak, the environmental information acquisition part further acquires environmental information including gas concentration around the gas leak position from at least one mobile second measurement part as additional environmental information, and
the operator identification part identifies, based further on the additional environmental information, the operator related to the gas leak from among the plurality of operators.

5. The management device according to claim 3,
wherein the operator identification part identifies an operator whose distance between a position identified by the position information and the gas leak position satisfies a predetermined distance range as the operator related to the gas leak.

6. The management device according to claim 5,
wherein in a case where a plurality of operators whose distance between the position identified by the position information and the gas leak position satisfies a predetermined distance range are identified by the operator identification part, the authentication part authenticates that each of the plurality of operators caused the gas leak at respective ratios identified according to a magnitude of the distance between the position of the plurality of operators and the gas leak position.

7. The management device according to claim 3, further comprising:
a facility information acquisition part acquiring equipment information corresponding to the gas leak position by referring to equipment information indicating information related to equipment that processes the gas as the measurement target and a position of the equipment; and
a facility information identification part identifying equipment information of at least one other equipment related to the equipment information corresponding to the gas leak position by referring to the equipment information,
wherein the environmental information acquisition part acquires environmental information around a position of the at least one other equipment from the at least one first measurement part, and
the estimation part estimates, based on past environmental information around the gas leak position and the environmental information around the position of the at least one other equipment, other equipment that may cause a gas leak within a predetermined period from among the at least one other equipment.

8. The management device according to claim 3,
wherein the estimation part estimates the gas leak position using an inverse analysis model, a statistical model, an atmospheric dispersion prediction model, or a machine learning model indicating a relationship between environmental information and a gas leak position.

9. The management device according to claim 3,
wherein the estimation part estimates the gas leak position using an inverse analysis model, a statistical model, an atmospheric dispersion prediction model, or a machine learning model indicating a relationship between the environmental information, which includes at least one of the gas concentration, wind direction, and wind speed of gas measured by the at least one first measurement part, and an estimated gas leak position tuned based on the environmental information.

10. The management device according to claim 3,
wherein the estimation part estimates, based on the environmental information, which includes at least one of the gas concentration, wind direction, and wind speed of gas measured by the at least one first measurement part, the gas leak position by calculating behavior of particles tracing back in time using a particle method.

11. The management device according to claim 3,
wherein the estimation part defines an objective function by comparing gas concentration at a reference point in a gas concentration distribution predicted using a machine learning model with gas concentration at the reference point measured by the at least one first measurement part, the machine learning model trained to predict a gas concentration distribution with the environmental information, which includes at least one of the gas concentration, wind direction, and wind speed of a gas measured by the at least one first measurement part as an explanatory variable and a gas concentration distribution as an objective variable; tunes the machine learning model such that the objective function becomes optimal; and estimates based on the gas concentration distribution predicted using the tuned machine learning model the gas leak position.

12. The management device according to claim 3,
wherein the environmental information further includes at least one of wind direction, wind speed, temperature and humidity, atmospheric pressure, solar radiation, and rainfall.

13. The management device according to claim 3,
wherein the at least one first measurement part comprises a gas sensor that is installed within a range of a vertical deployment angle of 60 degrees with reference to a height of a position where gas leak may occur and that measures concentration of the gas as the measurement target.

14. The management device according to claim 11,
wherein in a case where a ventilation time is defined as a time until 90% of an enclosure volume of the at least one first measurement part is ventilated by diffusion ventilation, the ventilation time of the at least one first measurement part is 60 seconds or less.

15. The management device according to claim 3, further comprising:
an issue part issuing a certificate indicating an authentication result by the authentication part,
wherein the issue part stores the certificate in a digital repository accessible and referable by a third party.

16. The management device according to claim 15,
wherein the issue part refers to the digital repository and issues the certificate further indicating a past authentication result identified based on a past certificate of the operator related to the gas leak.

17. The management device according to claim 3, further comprising:
a calibration information acquisition part acquiring calibration information including at least one of a calibration period at which calibration of the at least one first measurement part is to be executed, a calibration date and time at which calibration was executed by the at least one first measurement part, a calibration method, and calibrator information,
wherein in a case where the calibration information indicates that the at least one first measurement part is calibrated according to predetermined calibration conditions, the authentication part authenticates the identified operator related to the gas leak as an operator that caused the gas leak.

18. A management system, comprising the management device according to claim 1; and
the at least one first measurement part.

19. An authentication device, comprising:
a communication interface configured to be capable of communicating with at least one first measurement part;
an arithmetic device comprising at least one processor configured to be capable of communicating with the communication interface; and
a memory device storing a predetermined authentication protocol,
wherein the communication interface is configured to receive environmental information, which includes concentration of at least one type of measurement target gas from the first measurement part,
wherein the arithmetic device comprises:
an environmental information acquisition part acquiring the environmental information via the communication interface;
an estimation part estimating based on the environmental information an occurrence of leak of the measurement target gas and a position where the leak occurred as a gas leak position;
an operator identification part identifying, based on operator information, which includes a combination of an installation position of gas equipment and identification information of an operator managing the gas equipment as well as the gas leak position estimated by the estimation part, an operator related to the gas leak; and
an authentication part
authenticating based on the authentication protocol the identified operator related to the gas leak as an operator that caused the gas leak, and
issuing an electronic certificate for the operator authenticated by the authentication part and transmitting the electronic certificate via the communication interface.

20. A management method, comprising:
acquiring, by an environmental information acquisition part, environmental information from at least one first measurement part that measures the environmental information of a measurement target;
estimating, by an estimation part based on the environmental information a fluid leak position indicating a position where fluid leaked; and
identifying, by an operator identification part, based on operator information indicating identification information for identifying an operator and position information related to the operator as well as the fluid leak position, an operator related to the fluid leak.
